(19) United States
(12) Patent Application Publication
(10) Pub. No.: US 2013/0310902 A1
(43) Pub. Date: Nov. 21, 2013

(54) Apparatus for Maintaining Treatment of Peripheral Neuropathy

(71) Applicant: Bradford Richdale, Austin, TX (US)

(72) Inventor: Bradford Richdale, Austin, TX (US)

(21) Appl. No.: 13/475,748

(22) Filed: May 18, 2012

(51) Int. Cl. A61N 5/06

(52) U.S. Cl. 607/88

(57) ABSTRACT

An apparatus for maintaining treatment of peripheral neuropathy is disclosed. The apparatus includes a controller, a memory device coupled to the controller, and a radiation generator. The radiation generator is controlled by the controller to generate light pulses for irradiating a selected component of a human's body. The radiation generator includes a first group of light-emitting diodes (LEDs) to provide light pulses having a wavelength centered around approximately 640 nm, a second group of LEDs to provide light pulses having a wavelength centered around approximately 780 nm, and a third group of LEDs to provide light pulses having a wavelength centered around approximately 880 nm.

### BACKGROUND OF THE INVENTION

1. Technical Field

The present invention relates to phototherapy in general, and in particular to an apparatus for maintaining treatment of peripheral neuropathy using light pulses.

2. Description of Related Art

Phototherapy involves illuminating a patient's body with light pulses generated by suitable light sources in the visible and infrared ranges to provide various health benefits for the patient. The photons from the light pulses are absorbed by the patient through the patient's skin and various acupuncture points. Connective tissues within the patient's body further conduct the light to deeper tissues and organs within the patient's body. By taking advantage of optical properties of biological tissues, suitable wavelengths of light can be delivered to the patient such that some of the light can be absorbed and used by the patient's body to provide therapeutic effects for different medical purposes.

### SUMMARY OF THE INVENTION

In accordance with a preferred embodiment of the present invention, an apparatus for maintaining treatment of peripheral neuropathy includes a controller, a memory device coupled to the controller, and a radiation generator. The radiation generator is controlled by the controller to generate light pulses for irradiating a selected component of a human's body. The radiation generator includes a first group of light-emitting diodes (LEDs) to provide light pulses having a wavelength centered around approximately 640 nm, a second group of LEDs to provide light pulses having a wavelength centered around approximately 780 nm, and a third group of LEDs to provide light pulses having a wavelength centered around approximately 880 nm.

All features and advantages of the present invention will become apparent in the following detailed written description.

### BRIEF DESCRIPTION OF THE DRAWINGS

The invention itself, as well as a preferred mode of use, further objects, and advantages thereof, will best be understood by reference to the following detailed description of an illustrative embodiment when read in conjunction with the accompanying drawings, wherein:

FIG. 1 is a diagram of an apparatus for maintaining treatment of peripheral neuropathy, in accordance with a preferred embodiment of the present invention;

FIG. 2 is a block diagram of the apparatus from FIG. 1, in accordance with a preferred embodiment of the present invention;

FIGS. 3A-3B illustrate various time intervals for irradiation using different wavelength ranges; and

FIG. 4 is a graph of light pulses intensity versus time.

### DETAILED DESCRIPTION OF A PREFERRED EMBODIMENT

### I. Introduction

Phototherapy is the application of light from an artificial light source to stimulate therapeutic effects in a human body. Photons from the light source can be absorbed by the human body through skin and various acupuncture points. Light absorbed through acupuncture points is believed to be transported especially efficiently along body channels, which are generally referred to as meridians, within the body through a process similar to internal reflections of light within an optical fiber. These body channels are believed to be connective tissue protein fibers having specialized optical properties, including refractive indices η that are greater than the refractive indices η′ of surrounding tissues, organs and other body material (η′_{avg}≈1.4).

Only light in certain wavelength ranges will be transported efficiently through the above-mentioned body channels. Absorption of light transported in the body channels has the potential of increasing cell metabolism from a depressed level back to a normal level. Phototherapy activates cell membranes within a human body by increasing a membrane's natural electrical charge, sometimes referred to as membrane capacitance. A human body's natural electromagnetic field (biofield) helps in organizing molecular structures in repair, regeneration and reproduction of cells and cell components and serves as a signal communication system in regulation of metabolic processes. The biofield may also serve as a power grid to provide electrical and/or chemical energy to drive and control biochemical and biophysical enzyme reactions that are part of a metabolic process. One such process is the receipt and conversion of light in a body channel, the activation of cell enzymes, and the enhanced production of adenosine triphosphate (ATP) from the activated enzymes.

Each photon delivered to the vicinity of a body component is intended to produce one or more free electrons through photoelectric absorption and/or Compton scattering of the photon in its peregrinations through the body component and surrounding material. It was found, by analogy with the Einstein photoelectric effect in a metallic or crystalline material, that the photon energy E must be at least a threshold value Eₜₕᵣₑₛ, which lies in a range of about 1.3-3.1 eV, depending upon the atomic and/or molecular constituents of the selected body component and surrounding material, in order to produce at least one free electron as the photon undergoes scattering within the body. For example, a photon with a wavelength λ=500 nm has an associated energy of 2.48 eV, and the wavelength range 400 nm≦λ≦950 nm corresponds to an energy range 1.31 eV≦E≦3.10 eV.

However, not all photons with a photon energy E just above the threshold value Eₜₕᵣₑₛ will produce a free electron. One important parameter is the rate r at which photons or photon energy is being delivered to a unit area of a body surface per unit time during an exposure time interval. Experiments had indicated that energy density rates r within the range of 0.0013 Joules/cm²/sec and 0.02 Joules/cm²/sec, averaged over a time interval of 5-45 min, is an appropriate range for many body components for green light (λ≈550 nm), red light (λ≈2640 nm), white light and/or infrared light (λ≈880 nm). Delivery of photon energy at a rate lower than about 0.0013 Joules/cm²/sec will have some effect but will require much longer radiation application time than an application time of 5-45 min. On the other hand, when photon energy is being delivered at a rate greater than 0.02 Joules/cm²/sec, the delivered photon energy may saturate the body's ability to distribute the photon energy and may produce bums, ionization or other undesired local sensitization of the body.

Another important parameter is accumulated energy E_{accum} delivered per unit area for the session in which radiation is applied. Experiments had indicated that an accumulated energy density between 2.5 Joules/cm² and 20 Joules/cm² is an appropriate range for many body components. An accumulated energy density greater than 20 Joules/cm² may produce burns, ionization or other undesired local sensitization of the body.

The produced free electrons ultimately come to equilibrium with the body component and adjacent material within the body, by attachment to an atom or molecule that can support attachment by another electron or by association with a assembly of substantially-free electrons that are weakly bound by the general electronic background of the local atomic and molecular constituents of the body. These equilibrated electrons have transferred substantially all their initial kinetic energy to one or more molecules in or adjacent to the body component, thus providing energy to promote certain healing processes in the body.

### II. Apparatus

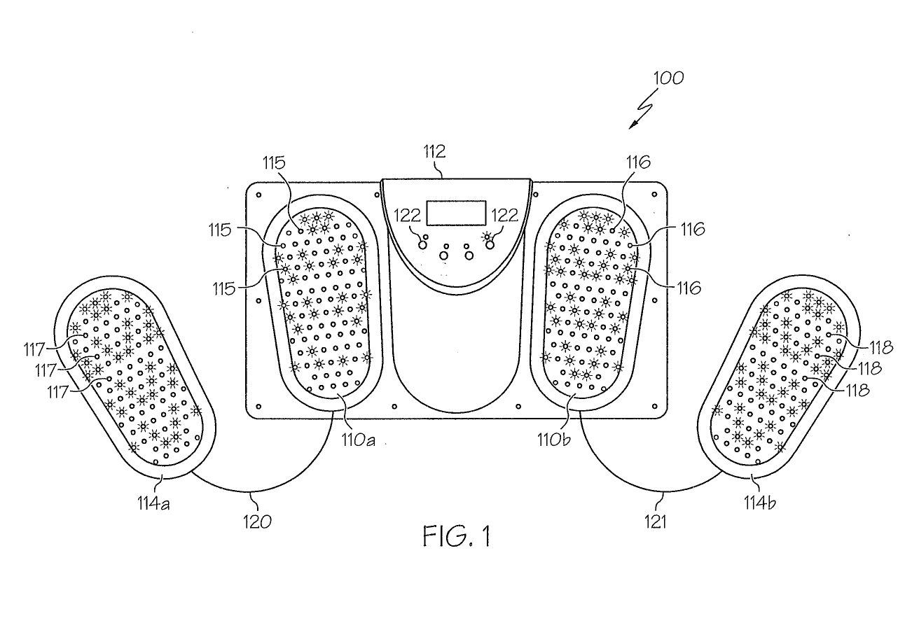

Referring now to the drawings and in particular to FIG. 1, there is depicted a diagram of an apparatus for maintaining treatment of peripheral neuropathy, in accordance with a preferred embodiment of the present invention. As shown, an apparatus 100 includes a display 112, a set of input buttons 122, a first pad 110a having an array of light-emitting diodes (LEDs) 115, and a second pad 110b having an array of LEDs 116. In addition, a third pad 114a having an array of LEDs 117 and a fourth pad 114b having an array of LEDs 118 can be optionally connected to apparatus 110 via wires 120, 121, respectively. Input buttons 122 allow a user to enter an operation mode for apparatus 100, and display 112 communicates various operational states of apparatus 100 to the user.

A user of apparatus 100 can place his/her feet on top of and in contact with first and second pads 110a, 110b to receive light pulses emitted from LEDs 115 and 116. Similarly, a user of apparatus 100 can place his/her hands on top of and in contact with third and fourth pads 114a, 114b to receive light pulses emitted from LEDs 117 and 118. The usage of first and second pads 110a, 110b and third and fourth pads 114a, 114b can be used simultaneously by one user.

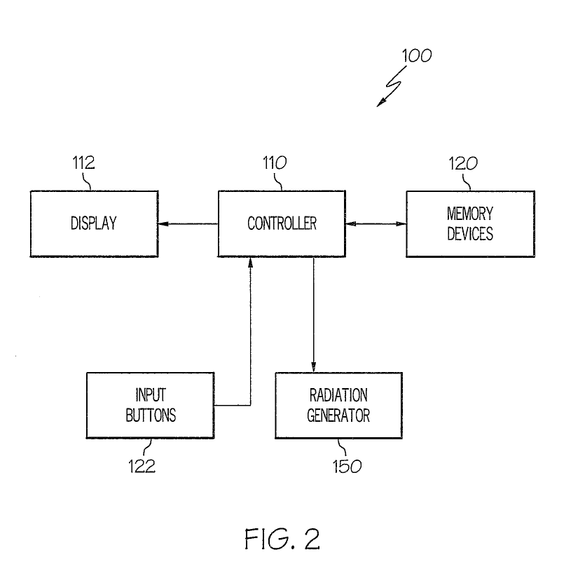

With reference now to FIG. 2, there is illustrated a block diagram of apparatus 100, in accordance with a preferred embodiment of the present invention. As shown, apparatus 100 includes a controller 110, memory devices 120, display 112, input buttons 122 and a radiation generator 150. Memory devices 120 preferably include a non-volatile storage device for storing a set of instructions to be executed by controller 110. Memory devices 120 also include volatile storage devices for receiving the set of instructions during their execution by controller 110. Display 112 provides various execution information such as modes of operation to a user.

Radiation generator 150 may produce a single or multiple beams of light that are intended to be directed to a human body. Radiation generator 150 is preferably made up of multiple LEDs, such as LEDs 115-118 shown in FIG. 1. However, it is understood by those skilled in the art that radiation generator 150 can be a laser, an intense incandescent light source, an intense fluorescent light source or any other suitable intense light source, or a combination of the above-mentioned light sources.

Radiation generator 150 is capable of generating electromagnetic radiation in the form of light in the visible and near-infrared ranges. Radiation generator 150 is capable of generating light pulses having wavelengths λ in the range between 415 nm and 900 nm. Radiation generator 150 is also capable of generating light pulses having wavelengths in the near-ultraviolet range between 350 nm and 400 nm, and in the mid-infrared range between 880 nm and 1,500 nm.

Controller 110 may activate (turn on) radiation generator 150 at selected time intervals, i.e., light-on time intervals Δtₒₙ, and may deactivate (turn off) radiation generator 150 at selected time intervals, i.e., light-off time intervals Δt_{off}, with one light-off time interval Δt_{off} inserted between two light-on time intervals Δtₒₙ or vice versa. Each of light-on time interval Δtₒₙ and light-off time interval Δt_{off} preferably lies between 0.5 second and 1.5 seconds.

The insertion of one light-off time interval Δt_{off} between two light-on time intervals Δtₒₙ is useful in allowing the irradiated body component to re-establish local equilibrium before the next pulse of photons arrives. The time interval required for re-establishing local equilibrium appears to vary from approximately 0.5 second to 1.5 seconds, depending upon variables such as the energy rate r, the accumulated energy E_{accum} and the selected body component being irradiated. If the light-off time interval is too short, the additional photons delivered may encounter a body environment that is not at or near equilibrium for channeling those photons in particular directions, which is generally undesirable. When two consecutive light-on time intervals are separated by an optimal light-off time interval, the irradiated body component is able to re-establish local equilibrium (or near-equilibrium) so that most, if not all, photons within a given exposure time interval encounter substantially the same local environment, and a random or Monte Carlo type of photon scattering occurs within the next exposure time interval.

In a preferred embodiment as depicted in FIG. 1, radiation generator 150 is implemented with LEDs 115-118. Since the arrangements of LEDs 115-118 on pads 110a, 110b, 114a and 114b, respectively, are substantially identical from each other, only the operations of LEDs 115 on pad 110a will be further described in details.

For the purpose of maintaining treatment of peripheral neuropathy, pad 110a includes three different groups of LEDs 115, each group being capable of delivering light of distinct wavelength as follows:
Group I—a broad band having λ centered around 640 nm;
Group II—a moderately broad band having λ centered around 780 nm; and
Group III—a narrow band having λ centered around 880 nm.

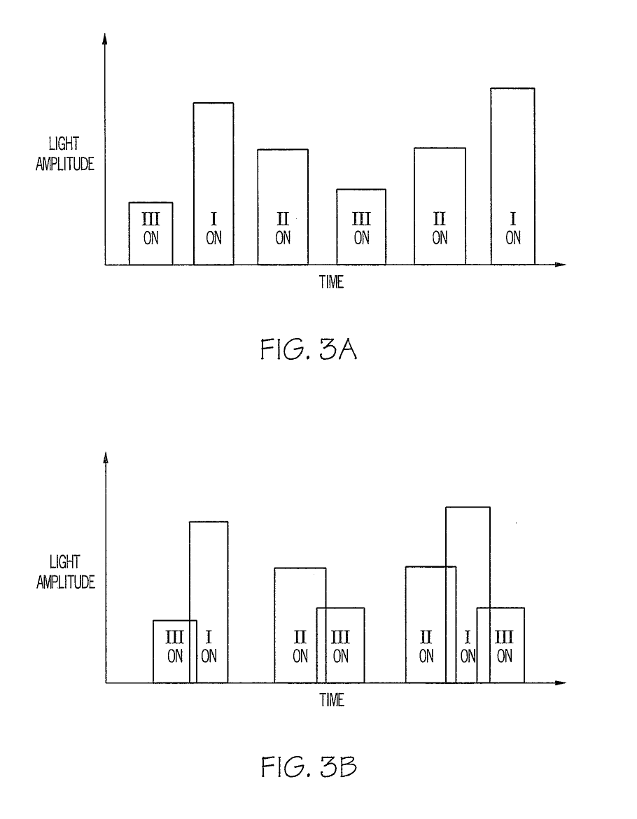

FIG. 3A depicts time intervals during which LEDs from Groups I, II and

III are being activated in a non-overlapping manner. The three groups of LEDs are being activated in the following sequence: III, I, II, III, II, I, III, I, II, III, II, I, etc.

FIG. 3B depicts time intervals during which LEDs from Groups I, II and III are being activated in an overlapping manner. The three groups of LEDs are being activated in the following sequence: III, I, II, III, II, I, III, I, II, III, II, I, etc.

Alternatively, each of LEDs 115 on pad 110a in FIG. 1 may deliver light in more than one wavelength ranges. For example, each of LEDs 115 is capable of delivering light in wavelength ranges between 400 nm and 550 nm and/or between 600 nm and 760 nm and/or between 800 nm and 1,500 nm.

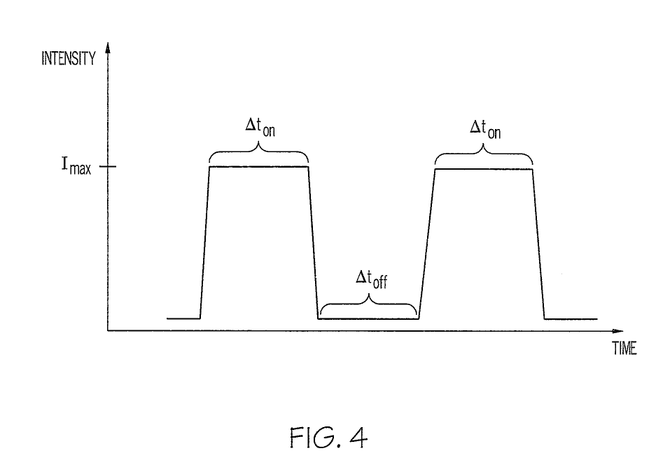

FIG. 4 illustrates representative light intensity patterns of light activation (lights on) and deactivation (lights off) that can be used for each of LEDs 115. As shown, the light intensity I begins at zero, then rises quickly to a maximum value Iₘₐₓ and stays on for approximately 0.5 to 1.5 seconds during a light-on time interval Δtₒₙ, then drops quickly to zero and stays off for approximately 0.5 to 1.5 seconds during a light-off time interval Δt_{off}, and then rises quickly to maximum value Iₘₐₓ and stays on for approximately 0.5 to 1.5 seconds during a light-on time interval Δtₒₙ. The above-mentioned pattern is repeated for preferably 45 minutes for one complete session of light treatment.

As has been described, the present invention provides a method and apparatus for illuminating body components with light pulses to provide therapeutic effects.

While the invention has been particularly shown and described with reference to a preferred embodiment, it will be understood by those skilled in the art that various changes in form and detail may be made therein without departing from the spirit and scope of the invention.

1. An apparatus for maintaining treatment of peripheral neuropathy, said apparatus comprising:
input buttons and a display;
a controller;
a memory device coupled to said controller; and
a radiation generator controlled by said controller to generate light pulses for irradiating a selected component of a human's body, wherein said radiation generator includes a first group of light-emitting diodes (LEDs) to provide light pulses having a wavelength centered around approximately 640 nm, a second group of LEDs to provide light pulses having a wavelength centered around approximately 780 nm, and a third group of LEDs to provide light pulses having a wavelength centered around approximately 880 nm.

2. The apparatus of claim 1, wherein said controller activates said radiation generator at a light-on time interval Δtₒₙ, and deactivates said radiation generator at a light-off time interval Δt_{off}, with one light-off time interval Δt_{off} inserted between two light-on time intervals Δtₒₙ or vice versa.

3. The apparatus of claim 2, wherein each of said light-on time interval Δtₒₙ and said light-off time interval Δt_{off} lies between approximately 0.5 second and 1.5 seconds.

4. The apparatus of claim 1, wherein light-on time intervals Δtₒₙ of each of said groups of LEDs do not overlap with each other.

5. The apparatus of claim 1, wherein light-on time intervals Δtₒₙ of some of said groups of LEDs overlap with each other.

6. The apparatus of claim 1, wherein said three groups of LEDs are being activated in the following sequence: said third group, said first group, said second group, said third group, said second group, said first group, etc.

7. The apparatus of claim 1, wherein said first group of LEDs provides broad band light pulses, said second group of LEDs provides mid band light pulses, and said third group of LEDs provides narrow band light pulses.

8. A method for maintaining treatment of peripheral neuropathy, said method comprising:
generating a first group of light pulses to irradiate a selected component of a human's body, wherein said first group of light pulses has a wavelength centered around approximately 640 nm;
generating a second group of light pulses to irradiate said selected component of a human's body, wherein said second group of light pulses has a wavelength centered around approximately 780 nm; and
generating a third group of light pulses to irradiate said selected component of a human's body, wherein said third group of light pulses has a wavelength centered around approximately 880 nm.

9. The method of claim 8, wherein said generating steps include a light-on time interval Δtₒₙ and a light-off time interval Δt_{off}, with one light-off time interval Δt_{off} inserted between two light-on time intervals Δtₒₙ or vice versa.

10. The method of claim 9, wherein each of said light-on time interval Δtₒₙ and said light-off time interval ≢t_{off} lies between approximately 0.5 second and 1.5 seconds.

11. The method of claim 9, wherein light-on time intervals Δtₒₙ of each of said groups of LEDs do not overlap with each other.

12. The method of claim 9, wherein light-on time intervals Δtₒₙ of some of said groups of LEDs overlap with each other.

13. The method of claim 8, wherein said three groups of LEDs are being activated in the following sequence: said third group, said first group, said second group, said third group, said second group, said first group, etc.

14. The method of claim 8, wherein said first group of light pulses is generated by a first group of LEDs, said second group of light pulses is generated by a second group of LEDs, and said third group of light pulses is generated by a third group of LEDs.
